(19) United States
(12) Patent Application Publication
(10) Pub. No.: US 2011/0112125 A1
(43) Pub. Date: May 12, 2011

(54) Novel hair growth composition

(75) Inventors: Yee-Chien Liu, Taipei County (TW); Pei-Lin Wu, Taipei County (TW)

(73) Assignee: Tritech Biopharmaceuticals Co., Ltd., Tainan (TW)

(21) Appl. No.: 12/590,401

(22) Filed: Nov. 9, 2009

(51) Int. Cl. A61K 8/49; A61Q 7/02

(52) U.S. Cl. 514/275

(57) ABSTRACT

A water-soluble minoxidil composition for topical use significantly enhances the transdermal delivery of the drug in comparison with that of Rogaine. The composition contains minoxidil, water, enhancer, and polymer. The percentage of water in the composition may be up to 95%.

### FIELD OF THE INVENTION

The present invention relates to a topical water-soluble minoxidil composition. In particular, the composition contains high percentage of water and almost organic solvent-free. The composition enhances the transdermal delivery of minoxidil, thereby significantly decreasing the risk of allergy.

### BACKGROUND OF THE INVENTION

Minoxidil is a blood vessel dilator which has been used in the treatment of cardiovascular diseases to lower blood pressure. The molecular mechanism how minoxidil works is still not fully understood; however, the activation of potassium channel via nitric oxide generation may play a pivotal role. Minoxidil is not only used in oral form to treat cardiovascular diseases but also formulated into a topical spray for the treatment of androgenetic alopecia (U.S. Pat. Nos. 4,596,812 and 4,139,619).

It has been over twenty years since minoxidil was used to treat alopecia. The popular formulations are tincture, foam, and gel, which contain 1-5% minoxidil by weight. In general, minoxidil dissolves poorly in both water and water-immiscible organic solvents. Therefore, the topical formulations of minoxidil must contain high percentages of ethanol and propylene glycol. For example, Rogaine extra strength (5% minoxidil) contains 30% ethanol and 50% propylene glycol. The ethanol-based vehicle, such as ethanol/propylene glycol/water, evaporates shortly after spreading over the bold skin; whereas, the greasy propylene glycol/water mixture stays on the applied area. Occasionally, the residual propylene glycol mixture causes allergic responses and results in dandruff, dermatitis, rash, and itchiness.

How to increase the solubility of minoxidil in water-based vehicles is an important subject in the field of transdermal delivery. U.S. Pat. Nos. 5,030,442 and 4,828,837 disclose that the inclusion of an amphiphatic compound with a pK less than 5 enhances the solubility of minoxidil. However, prolonged contact of the detergent-like amphiphatic compounds, such as N-methyl cocoyl taurate, laureth sulfosuccinate hemi-ester, lauryl sulfosuccinate, lysophosphatidic acid, monoalkyl phosphate ester, monoalkyl phosphonate, monoalkyl sulfonate, and oleamido-PEG-2-sulfosuccinate, may lead to allergic problems. Alternative strategies such as liposomes are used to formulate minoxidil (EP Pat. No. 0,177,223); however, formation of minoxidil crystalline might still occur when the composition is applied topically to skin as the vehicle evaporates.

### SUMMARY OF THE INVENTION

This invention is an object to formulate a water-soluble minoxidil composition for the treatment of hair loss and baldness. Since this water-soluble minoxidil composition is based on water and almost free of organic solvent, the allergic response caused by high concentration of propylene glycol and ethanol in the traditional minoxidil solution, such as Rogaine, may be significantly declined.

In addition, this composition also provides another advantage that the water-soluble formulation enhances the transdermal penetration of minoxidil in comparison with the effect of the traditional minoxidil composition.

This invention includes minoxidil, water, polymer, and enhancers. The weight ratio of minoxidil and polymer is from 4:1 to 1:4 and the composition has a preferred pH value from 4 to 5. The preferred polymers are carbomers, polyvinyl alcohols, polyacrylic acids, polyacrylates, polyvinyl pyrrolidones, polydextroses, cyclodextroses, polydextrins, cyclodextrins, polydextranes, and cyclodextranes.

This invention further contains at least one transdermal enhancer at a preferred ratio from 0.01% to 1%. The enhancers include NP-40, Tween-20, Tween-80, Triton X-100, Span-80, SDS, ethanol, propylene glycol, glycerol, PEG-400, cocamide betaine, coco imidazoline dicarboxylate, sodium lauroyl sarcosinate, sodium polyoxyethylene lauryl ether sulfate, potassium cocoyl glycinate, ammonium lauryl sulfate, coconut fatty acid diethanolamide, sorbitan monolaurate, sobitan sesquioleate, and sorbitol.

The water-soluble minoxidil composition is for topical use on skin, which may be administrated in gel, cream, lotion, ointment, or solution.

### BRIEF DESCRIPTION OF THE. DRAWINGS

FIG. 1 shows the HPLC analysis of minoxidil content in the hair-cut rat skins after the treatment of 5% Rogaine and various water-soluble minoxidil compositions for 1 hour. The skin tissues were either kept intact or decutinized via taping. Minoxidil in the treated areas was extracted and analyzed by HPLC. A: 1% water-soluble minoxidil composition; B: 2% water-soluble minoxidil composition; C: 3% water-soluble minoxidil composition; D: 4% water-soluble minoxidil composition; E: 5% water-soluble minoxidil composition; F: 5% Rogaine.

FIG. 2A shows the effect of various transdermal enhancers on minoxidil uptake after a 6-hr treatment. A: 5% Rogaine; B: 2% water-soluble minoxidil composition; C: 2% water-soluble minoxidil composition plus 0.5% Tween-20; D: 2% water-soluble minoxidil composition plus 1% cocamide betaine; E: 2% water-soluble minoxidil composition plus 2% sorbitol.

FIG. 2B shows the effect of various transdermal enhancers on minoxidil uptake after a 24-hr treatment. A: 5% Rogaine; B: 2% water-soluble minoxidil composition; C: 2% water-soluble minoxidil composition plus 0.1% NP-40; D: 2% water-soluble minoxidil composition plus 1% cocamide betaine.

### DETAILED DESCRPTION OF THE INVENTION

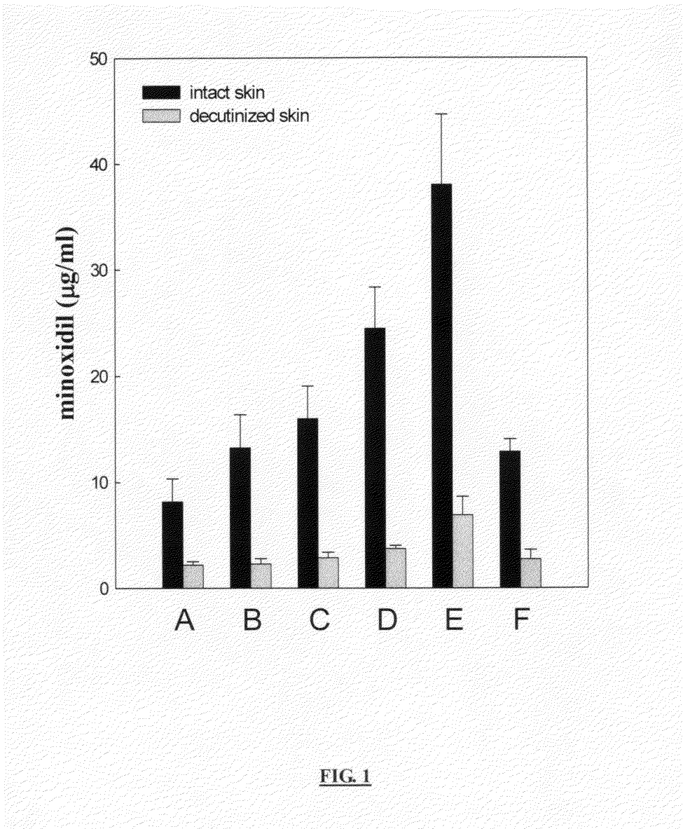

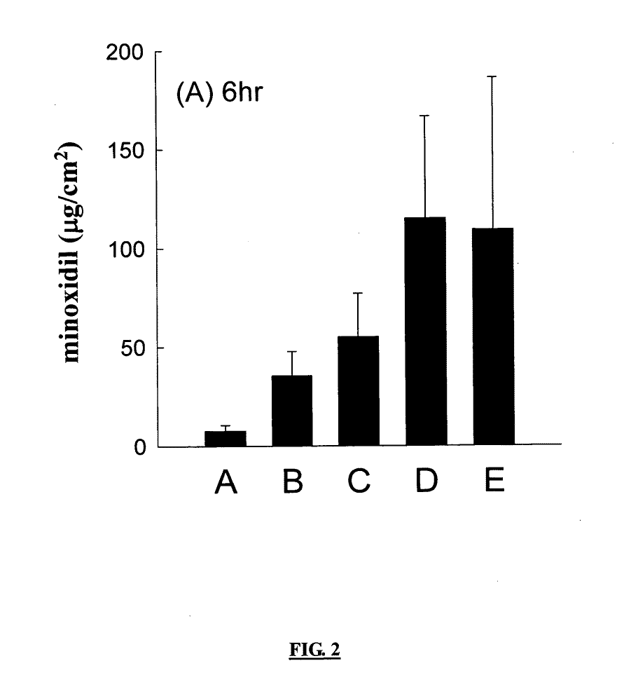

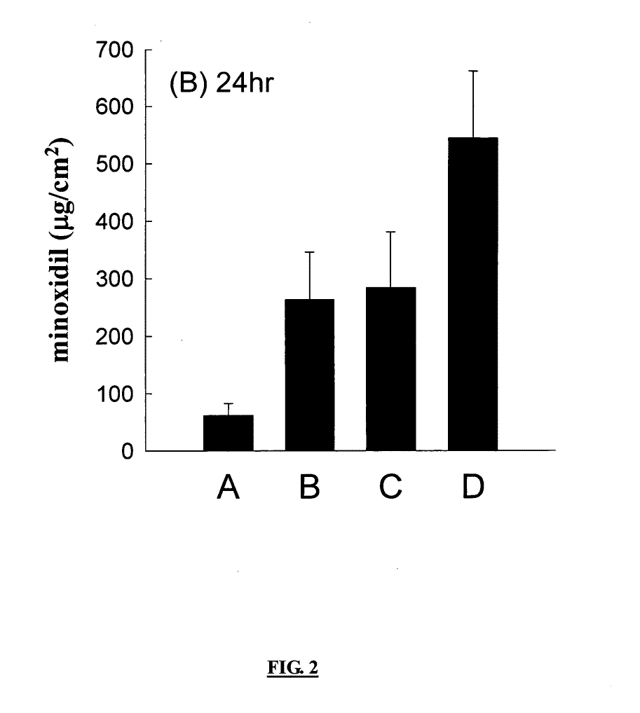

The water-soluble minoxidil composition is formulated by mixing minoxidil with polymers and enhancers. The polymers in the composition maintain the solubility of minoxidil in the water-based formula. The enhancers improve the transdermal efficacy of minoxidil in comparison with that of 5% Rogaine. In FIG. 1, the dorsal skins of rat were treated with various water-soluble minoxidil compositions or 5% Rogaine for 1 hour. The intact or decutinized skins were cut, and homogenized. The permeated minoxidil was extracted and analyzed by HPLC. The results indicated the 2% water-soluble minoxidil composition achieved the same penetrating ability as that of 5% Rogaine. In FIG. 2, the 2% water-soluble minoxidil composition, with or without an enhancer for either a 6-hour or 24-hour period, had a greater transdermal enhancement than that of 5% Rogaine. The present invention is further described below by referring to Examples; however, the present invention is not limited to these examples.

Water-soluble minoxidil compositions (1%-5%) are prepared according to the following material blending ratio and formulation. The “part” in the examples means “mass”

### EXAMPLES

### Example 1

Material blending ratio

| | |
|---|---|
| minoxidil | 1 part |
| Polyacrylic acid | 1 parts |
| H₂O | 95 parts |

### Formulation

Minoxidil is dispersed in water with continuous stirring for 10 min. Subsequently, 5M H₂SO₄ is added to dissolve minoxidil and the pH value is below 3. Polyacrylic acid is then dispersed into the solution and mix for additional 10 min. Finally, 5M NaOH is added to adjust the pH value between 4˜4.5. The weight ratio of H₂SO₄ and NaOH used in the formulation is from 1:2 to 2:1.

### Example 2

Material blending ratio

| | |
|---|---|
| minoxidil | 2 parts |
| polyvinyl alcohol | 2 parts |
| H₂O | 93 parts |

### Formulation

Minoxidil is dispersed in water with continuous stirring for 10 min. Subsequently, 5M H₂SO₄ is added to dissolve minoxidil and the pH value is below 3. Polyvinyl alcohol is then dispersed in water and stirred for 1 hour with heating at 80° C. Finally, two types of solutions are mixed and 5M NaOH is added to adjust the pH value between 4˜4.5. The weight ratio of H₂SO₄ and NaOH used in the formulation is from 1:2 to 2:1.

### Example 3

Material blending ratio

| | |
|---|---|
| minoxidil | 3 parts |
| beta-cyclodextrin | 2 parts |
| H₂O | 92 parts |

### Formulation

Minoxidil is dispersed in water with continuous stirring for 10 min. Subsequently, H₂SO₄ is added to dissolve minoxidil and the pH value is below 3. Beta-cyclodextrin is then dissolved in NaOH buffered water. Finally, two types of solutions are mixed and the pH value of the mixture is adjusted between 4˜4.5. The weight ratio of H₂SO₄ and NaOH used in the formulation is from 1:2 to 2:1.

### Example 4

Material blending ratio

| | |
|---|---|
| minoxidil | 4 parts |
| polyvinyl pyrrolidone (K90) | 3 parts |
| H₂O | 90 parts |

### Formulation

Minoxidil is dispersed in water with continuous stirring for 10 min. Subsequently, 5M H₂SO₄ is added to dissolve minoxidil and the pH value is below 3. Polyvinyl pyrrolidone is then dispersed into the solution and mix for additional 10 min. Finally, 5M NaOH is added to adjust the pH value between 4˜4.5. The weight ratio of H₂SO₄ and NaOH used in the formulation is from 1:2 to 2:1.

### Example 5

Material blending ratio

| | |
|---|---|
| minoxidil | 5 parts |
| polydextrose | 5 parts |
| H₂O | 87 parts |

### Formulation

Minoxidil is dispersed in water with continuous stirring for 10 min. Subsequently, 5M H₂SO₄ is added to dissolve minoxidil and the pH value is below 3. Polydextrose is then dispersed into the solution and mix for additional 10 min. Finally, 5M NaOH is added to adjust the pH value between 4˜4.5. The weight ratio of H₂SO₄ and NaOH used in the formulation is from 1:2 to 2:1.

What is claimed is:

1. A hair growth composition comprising:
(a) at least one active ingredient,
(b) at least one transdermal enhancer,
(c) at least one polymer, and
(d) water.

2. The composition of claim 1 wherein said composition is in a form of gel, cream, lotion, ointment, or solution.

3. The composition of claim 1 wherein said active ingredient is minoxidil.

4. The composition of claim 1 wherein said transdermal enhancer is selected from the group consisting of NP-40, Tween-20, Tween-80, Triton X-100, Span-80, SDS, ethanol, propylene glycol, glycerol, PEG-400, cocamide betaine, coco imidazoline dicarboxylate, sodium lauroyl sarcosinate, sodium polyoxyethylene lauryl ether sulfate, potassium cocoyl glycinate, ammonium lauryl sulfate, coconut fatty acid diethanolamide, sorbitan monolaurate, sobitan sesquioleate, and sorbitol.

5. The composition of claim 1 wherein said polymer is selected from the group consisting of carbomer, polyvinyl alcohol, polyacrylic acid, polyacrylate, polyvinyl pyrrolidone, polydextrose, cyclodextrose, polydextrin, alpha-cyclodextrin, beta-cyclodextrin, gamma-cyclodextrin, polydextrane, and cyclodextrane.

6. The composition of claim 3 wherein the concentration of minoxidil is from 1% to 5%.

7. The composition of claim 4 wherein the concentration of enhancer is from 0.01% to 2%.

8. The composition of claim 4 wherein the concentration of enhancer is from 0.05% to 1%.

9. The composition of claim 5 wherein the concentration of polymer is from 0.5% to 10%.

10. The composition of claim 5 wherein the concentration of polymer is from 1% to 5%.

11. The composition of claim 1 wherein said water is from 60% to 96%.
